(19) United States
(12) Patent Application Publication
(10) Pub. No.: US 2023/0146841 A1
(43) Pub. Date: May 11, 2023

(54) TNFAIP3 AS A BIOMARKER FOR AUTOIMMUNE DISEASES

(71) Applicant: The Brigham and Women's Hospital, Inc., Boston, MA (US)

(72) Inventor: Tanuja Chitnis, Chestnut Hill, MA (US)

(21) Appl. No.: 17/913,509

(22) PCT Filed: Mar. 23, 2021
(86) PCT No.: PCT/US2021/023611

§ 371 Date: Sep. 22, 2022

Related U.S. Application Data
(60) Provisional application No. 62/993,645, filed on Mar. 23, 2020.

(51) Int. Cl. C12Q 1/686; G01N 33/68

(52) U.S. Cl. CPC C12Q 1/686; G01N 33/6896; C12Q 2600/118; G01N 2800/285

(57) ABSTRACT

Aspects of the present disclosure relate to methods for detecting tumor necrosis factor alpha-induced protein 3 (TN-FAIP3) in a sample from a subject having or at risk for myelin oligodendrocyte glycoprotein antibody associated diseases (MOG-AAD) or a relapse thereof.

### CLAIM OF PRIORITY

This application claims the benefit of U.S. Provisional Patent Application No. 62/993,645, filed on Mar. 23, 2020, which is incorporated by reference herein in its entirety.

### FIELD OF THE INVENTION

The subject matter disclosed herein generally relates to methods of using tumor necrosis factor alpha-induced protein 3 (TNFAIP3) as a biomarker, e.g., as a biomarker for myelin oligodendrocyte glycoprotein antibody associated diseases (MOG-AAD).

### BACKGROUND OF THE INVENTION

Myelin oligodendrocyte glycoprotein antibody associated diseases (MOG-AAD) have been recently described in approximately 25% of children and 5% of adults with demyelinating disorders. MOG is a component of myelin expressed in the central nervous system (CNS), and MOG antibodies can be detected by cell-based assays in the serum. MOG-AAD exhibits an age-related array of clinical phenotypes ranging from acute disseminated encephalomyelitis (ADEM), clinically isolated syndrome (CIS), optic neuritis (ON), recurrent forms of ADEM and ON, transverse myelitis (TM) and neuromyelitis spectrum disorder (NMO-SD). Approximately 50% of patients experience a multiphasic disease course. There is an increasing body of literature demonstrating that patients with MOG-AAD have distinct clinical and MRI features from multiple sclerosis (MS). Treatment response varies between MOG-AAD patients, and immunosuppressive treatments including rituximab or mycophenolate mofetil often result in incomplete control of disease. Moreover, standard multiple sclerosis medications such as beta interferon may exacerbate disease.

Individual patients with MOG-antibodies experience varying disease courses, some with frequent relapses, which can result in significant disability. One of the main challenges in clinical care is to identify patients who will develop a multiphasic disease course, and require chronic immunotherapy. The persistent presence of high titers of serum MOG antibody titers has been studied in the prognostication of a multiphasic disease and relapses, although with inconsistent results. For these collective reasons, identifying a MOG-AAD-specific biomarker with diagnostic and prognostic relevance has emerged as a major objective in the field.

### SUMMARY OF THE INVENTION

The present disclosure is based, at least in part, on the finding that reduced levels of TNFAIP3 can be found in blood samples from MOG-AAD patients at relapse as compared to blood samples from MOG-AAD patients at remission or blood samples from control patients (e.g., healthy patients).

Accordingly, aspects of the present disclosure provide methods for analyzing a sample comprising: providing a sample from a subject, and detecting tumor necrosis factor alpha-induced protein 3 (TNFAIP3) in the sample.

In some embodiments, detecting TNFAIP3 in the sample comprises detecting a level of TNFAIP3 protein in the sample. In some embodiments, the level of TNFAIP3 protein is detected by an immunohistochemical assay, an immunoblotting assay, or a flow cytometry assay.

In some embodiments, detecting TNFAIP3 comprises detecting a level of TNFAIP3 nucleic acid. In some embodiments, the level of TNFAIP3 nucleic acid is detected by a real-time reverse transcriptase PCR (RT-PCR) assay or a nucleic acid microarray assay.

In some embodiments, the sample is a blood sample or a serum sample. In some embodiments, the sample is obtained from a subject having or suspected of having myelin oligodendrocyte glycoprotein antibody associated diseases (MOG-AAD). In some embodiments, the MOG-AAD is selected from the group consisting of acute disseminated encephalomyelitis (ADEM), clinically isolated syndrome (CIS), optic neuritis (ON), recurrent forms of ADEM and ON, transverse myelitis (TM), and neuromyelitis spectrum disorder (NMO-SD).

In some embodiments, methods further comprise administering the subject a treatment for MOG-AAD. In some embodiments, the subject is a human patient or a non-human animal.

Aspects of the present disclosure provide methods of diagnosing a subject as having myelin oligodendrocyte glycoprotein antibody associated diseases (MOG-AAD) or a relapse thereof comprising: providing a sample from the subject, detecting a level of tumor necrosis factor alpha-induced protein 3 (TNFAIP3) in the sample, comparing the level of TNFAIP3 in the sample to a reference level, and diagnosing a subject as having MOG-AAD or a relapse thereof when the level of TNFAIP3 in the sample is lower than the reference level.

In some embodiments, detecting TNFAIP3 in the sample comprises detecting a level of TNFAIP3 protein in the sample. In some embodiments, the level of TNFAIP3 protein is detected by an immunohistochemical assay, an immunoblotting assay, or a flow cytometry assay.

In some embodiments, detecting TNFAIP3 comprises detecting a level of TNFAIP3 nucleic acid. In some embodiments, the level of TNFAIP3 nucleic acid is detected by a real-time reverse transcriptase PCR (RT-PCR) assay or a nucleic acid microarray assay.

In some embodiments, the sample is a blood sample or a serum sample. In some embodiments, the sample is obtained from a subject having or suspected of having MOG-AAD or a relapse thereof. In some embodiments, the MOG-AAD is selected from the group consisting of acute disseminated encephalomyelitis (ADEM), clinically isolated syndrome (CIS), optic neuritis (ON), recurrent forms of ADEM and ON, transverse myelitis (TM), and neuromyelitis spectrum disorder (NMO-SD).

In some embodiments, methods further comprise administering the subject a treatment for MOG-AAD. In some embodiments, the subject is a human patient or a non-human animal.

### BRIEF DESCRIPTION OF THE DRAWINGS

FIGS. 1A-ID include data showing cytokine expression in MOG-reactive central memory cells (CMCs) from untreated MOG-AAD patients and pediatric healthy controls (PHCs): PBMCs were stimulated and cultured with 10 μg/ml of MOG peptides (p1-20, p35-55, p119-130, p181-195 and p186-200) and cocktail of myelin peptides (Myelin Phospholipid Protein 139-154 (PLP), Myelin Basic Protein (MBP) 13-32, MBP 111-129 and MBP 146-170) for 7 days followed by staining with antibodies and flow cytometry as described in Materials and Methods. Data was analyzed using Flowjo vX.0.7 and the graphs were made using GraphPadPrism version 8.4.2 (464). FIG. 1A includes graphs showing percentage of CMCs (CD4+CCR7+CD45RA−). FIG. 1B includes graphs showing percentage of IL17+producing CMCs (CD4+CCR7+CD45RA−, IL17+). FIG. 1C includes graphs showing percentage of IFNγ+producing CMCs (CD4+CCR7+CD45RA−, IFNγ+). FIG. 1D includes graphs showing percentage of IL17+ and IFNγ+producing CMCs (CD4+CCR7+CD45RA−, IL17+, IFNγ+). MOG-AAD n=8, PHC n=7, Mann-Whitney test, *P<0.05.

FIGS. 2A-2D include data showing cytokine expression in MOG-reactive central memory cells (CMCs) from MOG-AAD patients at the time of relapse and remission: PBMCs were stimulated and cultured with 10 μg/ml of MOG peptides (p1-20, p35-55, p119-130, p181-195 and p186-200) and cocktail of myelin peptides (Myelin Phospholipid Protein 139-154, Myelin Basic Protein (MBP) 13-32, MBP 111-129 and MBP 146-170) for 7 days followed by staining with antibodies and flow cytometry as described in Materials and methods. Data was analyzed using Flowjo vX.0.7 and the graphs were made using GraphPadPrism version 8.4.2 (464). FIG. 2A includes graphs showing percentage of IL17+producing CMCs (CD4+CCR7+CD45RA−, IL17+). FIG. 2B includes graphs showing percentage of IFNγ+producing CMCs (CD4+CCR7+CD45RA−, IFNγ+). FIG. 2C includes graphs showing percentage of IL17+ and IFNγ+producing CMCs (CD4+CCR7+CD45RA−, IL17+, IFNγ+). Relapse n=9, Remission n=6 FIG. 2D includes graphs showing percentage CMCs (CD4+CCR7+CD45RA−), CMC IL17+(CD4+CCR7+CD45RA−, IL17+), CMC IFNγ+(CD4+CCR7+CD45RA−, IFNγ+), CMC++(CD4+CCR7+CD45RA−, IL17+, IFNγ+) analysis from a single pediatric MOG-AAD patient #1 with longitudinal samples, (1) Rm (remission, MOG-AAD #1.2), (2) PR (pre-relapse, MOG-AAD #1.3), (3) R (relapse, MOG-AAD #1.4) and (4) Rm/S (remission treated with steroid, MOG-AAD #1.5).

FIGS. 3A-3D include data showing gene expression analysis in PBMCs from MOG-AAD patients. FIG. 3A includes graphs showing TNFAIP3 and NFκβ1 expression by DGE sequencing. NanoString Gene Expression Assay was performed on a MOG-AAD patient #2 with 3 longitudinal samples, (1) untreated R (relapse, MOG-AAD #2.1), (2) 8 months Rm/Ct (mycophenolate mofetil treated at remission, MOG-AAD #2.2), and (3) 11 months Rm/Ct (mycophenolate mofetil treated at remission, MOGAAD #2.3) as described in Materials and methods. Data were normalized and analyzed using nSolver software via the geometric mean of included housekeeping genes. The graphs were made using GraphPadPrism version 8.4.2 (464). FIG. 3B includes graphs showing TNFAIP3 and TNF-α expression by NanoString Gene Expression Assay. qPCR was performed on CD4+ T cells from 7 MOG-AAD patients with longitudinal samples as described in Materials and methods. It also included MOG-AAD patient #2 with 3 longitudinal samples as previously used for NanoString gene expression assay. The graphs were made using GraphPadPrism version 8.4.2 (464). FIG. 3C includes graphs showing TNFAIP3 expression in MOG-AAD patient #2 by qPCR. FIG. 3D includes graphs showing grouped analysis of TNFAIP3 expression in relapse samples, remission samples and samples treated with corticosteroids by qPCR. Relapse n=5, Remission n=5, Steroid n=4, Ordinary 1-way ANOVA; P=0.0137.

FIGS. 4A-4B include data showing TNFAIP3 protein expression in relapse and non-relapse samples from an untreated MOG-AAD patient: PBMCs from a MOG-AAD patient #3 at a relapse (MOG-AAD #3.1) and non-relapse (MOG-AAD #3.2) time point were cultured under different conditions, analyzed by SDS-PAGE and followed by western blot for TNFAIP3 at the indicated time points (hours). FIG. 4A includes data showing MOG antigen stimulation at 1 μg/ml (peptide cocktail comprising of MOG p1-20, p35-55, p119-130, p181-195 and p186-200). FIG. 4B includes data showing MOG antigen+Dexamethasone stimulation at 1 μg+100 nm. Representative Western blot images are shown on left and protein bands quantified using ImageJ version 1.53b and normalized to 3-actin are shown on the right.

FIGS. 5A-5F include data showing serum levels of TNFAIP3 in indicated longitudinal samples of MOG-AAD patients determined by ELISA. FIG. 5A includes a graph of TNFAIP3 levels in MOG-AAD patient #1 with 5 longitudinal samples, (1) Rm (remission, MOG-AAD #1.1), (2) Rm (remission, MOGAAD #1.2), (3) PR (pre-relapse, MOG-AAD #1.3), (4) R (relapse, MOG-AAD #1.4), and (5) Rm/S (remission treated with steroid, MOG-AAD #1.5). FIG. 5B includes a graph of TNFAIP3 levels in MOG-AAD patient #5 with 8 longitudinal samples, (1) Rm (remission, MOG-AAD #5.1), (2) Rm (remission, MOG-AAD #5.2), (3) Rm (remission, MOGAAD #5.3), (4) Rm (remission, MOG-AAD #5.4), (5) PR (pre-relapse, MOG-AAD #5.5), (6) Rm (remission, MOG-AAD #5.6), (7) Rm/Ct (mycophenolate mofetil treated at remission, MOG-AAD #5.7), and (8) Rm/Ct (mycophenolate mofetil treated at remission, MOG-AAD #5.8). FIG. 5C includes a graph of TNFAIP3 levels in MOG-AAD patient #6 with 3 longitudinal samples, (1) R/S (relapse treated with steroid, MOG-AAD #6.1), (2) Rm/Ct (mycophenolate mofetil treated at remission, MOG-AAD #6.2), and (3) Rm/Ct (mycophenolate mofetil treated at remission, MOG-AAD #6.3). FIG. 5D includes a graph of TNFAIP3 levels in MOG-AAD patient #7 with 2 longitudinal samples, (1) R/S (relapse treated with steroid, MOG-AAD #7.1), and (2) Rm/Rebif (remission treated with interferon beta-1a, MOGAAD #7.2). FIG. 5E includes a graph of TNFAIP3 levels in MOG-AAD patient #8 with 2 longitudinal samples, (1) Rm (remission, MOGAAD #8.1) and (2) R/S/Co (remission treated with steroid and Glatiramer acetate, MOG-AAD #8.2). FIG. 5F includes a graph of mixed model paired analysis compared MOG-AAD patients, who had, both, relapse and remission samples. Relapse n=8, Remission n=22. The comparison between the relapse and remission group was done using Nonlinear Mixed-Effects Models library in R (nlme).

The details of one or more embodiments of the invention are set forth in the description below. Other features or advantages of the present invention will be apparent from the following drawings and detailed description of several embodiments, and also from the appended claims.

### DETAILED DESCRIPTION

Aspects of the present disclosure relate to methods for detecting TNFAIP3 in a sample (e.g., a serum sample) from a subject (e.g., a patient) with MOG-AAD, e.g., a subject with MOG-AAD who is having a relapse or at risk for a relapse. Non-limiting examples of MOG-AAD include acute disseminated encephalomyelitis (ADEM), clinically isolated syndrome (CIS), optic neuritis (ON), recurrent forms of ADEM and ON, transverse myelitis (TM), or neuromyelitis spectrum disorder (NMO-SD).

Such methods can be useful for clinical purposes, e.g., identifying a subject having or at risk for MOG-AAD, identifying a subject having or a risk for a relapse of MOG-AAD, selecting a treatment for MOG-AAD, monitoring MOG-AAD progression and/or monitoring MOG-AAD relapse, assessing the efficacy of a treatment against MOG-AAD, or determining a course of treatment for a subject.

The assay methods described herein can also be useful for non-clinical applications, e.g., for research purposes, including, e.g., studying the mechanism of MOG-AAD development and/or biological pathways and/or biological processes involved in MOG-AAD, and developing new therapies for MOG-AAD based on such studies.

Methods described herein are based, at least in part, on the identification of TNFAIP3 as a biomarker for relapse in patients with MOG-AAD. As used herein, the term “biomarker” refers to a biological molecule that is present at a level in a subject that deviates from a level of the same biological molecule in a different subject. For example, TNFAIP3 that is indicative of a relapse of MOG-AAD can have a reduced level in a sample from a subject (e.g., a serum sample from a subject that has or is at risk for a relapse of MOG-AAD) relative to the level of TNFAIP3 in a control sample (e.g., a serum sample from a subject with MOG-AAD who is not having or at risk for a relapse such as a subject in remission or a healthy subject such as a subject who does not have or is not at risk for MOG-AAD).

In studies described herein, PBMC and serum samples from MOG-AAD patients at relapse and remission time points were analyzed by transcriptomic analysis. In central memory T cells (CMC), TNFAIP3 gene expression was decreased during a relapse, and this decrease correlated with a subsequent activation of NFκβ signaling. Protein analysis demonstrated that MOG-AAD relapse and remission samples respond differently to antigen stimulation, suggesting a dysregulation of TNAIP3 response, which may be dependent on cell state. It was also found that TNFAIP3 serum level decreases are associated with the onset of a relapse in individual MOG-AAD patients, and thus TNFAIP3 levels can function as a biomarker and therapeutic target for MOG-AAD and other autoimmune diseases.

The human TNFAIP3 gene is located on chromosome 6, and is also known as A20. It encodes the 790 amino acid protein, A20 that is, made of an N-terminal protease domain and seven Cys2-Cys2 zinc finger C-terminal domains. A20 is an ubiquitin-editing enzyme belonging to the ovarian tumor (OTU) proteases family of deubiquitinating (DUB) enzymes. A20 functions as an E3 ubiquitin ligase as a result of its fourth zinc finger motif in the C-terminal domain TNFAIP3 is a key regulator of cellular processes including NFκβ activation and apoptosis. TNFAIP3 suppresses cellular processes by down regulating NFκβ activation in part through DUB activity, ubiquitin-binding activity, and/or E3 ligase activity of critical signaling components including RIP1, TRAF6 and NEMO, upstream of the IKK complex. It also binds directly to the C-terminus of IL-17RA, and can decrease IL-17 responses through inhibition of p38. Several stimuli, including TNFα, LPS, TLR and IL-1 through activation of NFκβ via the non-canonical pathway, increase TNFAIP3 mRNA expression, thus initiating a negative feedback loop to regulate NFκβ via TRAF1/TRAF2. TNFAIP3-deficient cells fail to terminate TNF-α-induced NFκβ responses. Mice deficient in TNFAIP3 develop severe cachexia and inflammation in the liver, kidneys, intestines, joints, and bone marrow, and die prematurely.

Activation of NFκβ through the canonical pathway related to TCR activation requires MALT1 signaling, which is suppressed by TNFAIP3 through deubiquitination. In turn, MALT1 mediates rapid proteolytic cleavage and inactivation of TNFAIP3 after TCR stimulation, thus fine-tuning TCR activation. MALT1 is also required for BCR activation and TNFAIP3 deletion is frequently observed in B cell lymphomas.

Results described herein demonstrated that CD4+ T cell and serum decrements of TNFAIP3 are associated with clinical relapses in MOG-AAD patients and also associated with relative increases in NF-κβ expression, which is consistent with the role of TNFAIP3 in modulating NF-κβ activation. However, these results could be due to the effects of strong antigen-specific activation of T cells in MHC-matched patients, or because of aberrant regulation of TNFAIP3 in this condition. Indeed, genetic polymorphisms of the TNFAIP3 gene have been described in several autoimmune diseases including rheumatoid arthritis, psoriasis, type 1 diabetes, inflammatory bowel disease, systemic lupus erythematosus (SLE), coronary artery disease and celiac disease. Several TNFAIP3 intergenic polymorphisms have also been associated with MS susceptibility. MOG-AAD is closely related to MS, however it has distinct clinical and radiological features. A comprehensive large-scale genomic analysis of MOG-AAD has not been reported thus far.

MOG-AAD, which is largely a pediatric disease, is highly sensitive to glucocorticoid treatment. Glucocorticoids bind to the glucocorticoid receptor (GR) leading to immune suppression. TNFAIP3, which is an anti-inflammatory target of TNF-α, inhibitor of NF-κβ, is regulated by steroids including estrogen and glucocorticoids. While stimulation with MOG antigen failed to induce TNFAIP3, dexamethasone stimulation, a potent glucocorticoid, partially rescued the expression of TNFAIP3 protein and transcriptome levels in the relapse sample, thereby suggesting that the induction of TNFAIP3 by glucocorticoids can improve relapses in MOG-AAD patients.

Serum TNFAIP3 levels may be related to secretion by immune cells. The decrement in TNFAIP3 may be associated with antigen-stimulation of the TCR, thus associated with activation of antigen-specific T cells. In studies described herein, gene expression was analyzed only in CMC T cells, which are integral in initiating antigen-specific immune response. However, B cells, monocytes and other cells also express TNFAIP3, and may be involved in eliciting TNFAIP3 serum levels. The serum level of TNFAIP3 varies between individual patients, and only decrements observed in longitudinal samples, but not absolute values were associated with relapses.

### I. Methods for Detection of TNFAIP3

Any sample that may contain TNFAIP3 can be analyzed by the assay methods described herein. The methods described herein involve providing a sample obtained from a subject. In some examples, the sample may be from an in vitro assay, e.g., from an in vitro cell culture (e.g., an in vitro cell culture of CD4+ T cells). In other examples, the sample to be analyzed by the assay methods described herein is a biological sample.

As used herein, a “sample” refers to a composition that comprises biological materials including, but not limited to, blood, plasma, serum, tissue, cells, and/or fluid from a subject. A sample includes both an initial unprocessed sample taken from a subject as well as subsequently processed, e.g., partially purified or preserved forms. In some embodiments, the sample is plasma. In some embodiments, multiple (e.g., at least 2, 3, 4, 5, or more) samples may be collected from a subject, over time or at particular time intervals, for example, to assess relapse or progression of the disease or evaluate the efficacy of a treatment. A sample can be obtained from a subject using any means known in the art.

The term “subject” or “patient” can be used interchangeably and refers to a subject who needs the analysis as described herein. In some embodiments, the subject is a human or a non-human mammal (e.g., cat, dog, horse, cow, goat, or sheep). In some embodiments, a subject is suspected of or is at risk for MOG-AAD or a relapse thereof. Such a subject can exhibit one or more symptoms associated with MOG-AAD or a relapse thereof. Alternatively or in addition to, such a subject can have one or more risk factors for MOD-AAD or a relapse thereof, e.g., genetic susceptibility such as certain HLA subtypes.

Alternatively, the subject who needs the analysis described herein can be a patient having MOG-AAD. Such a subject can currently be having a relapse, or can have suffered from the disease in the past (e.g., currently relapse-free). In some examples, the subject is a human patient who can be on a treatment of the disease, e.g., a treatment involving an immunosuppressive agent (e.g., mycophenolate mofetil, azathioprine). In other instances, such a human patient can be free of such a treatment.

Examples of MOG-AAD include, without limitation, acute disseminated encephalomyelitis (ADEM), clinically isolated syndrome (CIS), optic neuritis (ON), recurrent forms of ADEM and ON, transverse myelitis (TM), and neuromyelitis spectrum disorder (NMO-SD).

Any sample (e.g., those described herein) can be used in the methods described herein, which involve measuring the level of TNFAIP3 as described herein. Levels (e.g., the amount) of TNFAIP3 disclosed herein, or changes in levels of TNFAIP3, can be assessed using conventional assays or those described herein.

As used herein, the terms “measuring” or “measurement,” or alternatively “detecting” or “detection,” means assessing the presence, absence, quantity or amount (which can be an effective amount) of TNFAIP3 within a sample, including the derivation of qualitative or quantitative concentration levels of TNFAIP3, or otherwise evaluating the values and/or categorization of TNFAIP3 in a sample from a subject.

In some embodiments, the level of TNFAIP3 is assessed or measured by directly detecting TNFAIP3 protein in a sample (e.g., a serum sample). Alternatively, or in addition to, the level of TNFAIP3 protein can be assessed or measured indirectly in a sample, for example, by detecting the level of activity of TNFAIP3 protein.

The level of TNFAIP3 protein can be measured using an immunoassay. Examples of immunoassays include any known assay (without limitation), and can include any of the following: immunoblotting assay (e.g., Western blot), immunohistochemical analysis, flow cytometry assay, immunofluorescence assay (IF), enzyme-linked immunosorbent assays (ELISAs) (e.g., sandwich ELISAs), radioimmunoassays, electrochemiluminescence-based detection assays, magnetic immunoassays, lateral flow assays, and related techniques. Additional suitable immunoassays for detecting TNFAIP3 protein will be apparent to those of skill in the art.

An exemplary sequence of human TNFAIP3 protein is provided in GenBank as Accession No. NP_006281.1, e.g., as follows:

| (SEQ ID NO: 1) | |
|---|---|
| 1 | MAEQVLPQAL YLSNMRKAVK IRERTPEDIF KPTNGIIHHF KTMHRYTLEM FRTCQFCPQF |
| 61 | REIIHKALID RNIQATLESQ KKLNWCREVR KLVALKTNGD GNCLMHATSQ YMWGVQDTDL |
| 121 | VLRKALFSTL KETDTRNFKF RWQLESLKSQ EFVETGLCYD TRNWNDEWDN LIKMASTDTP |
| 181 | MARSGLQYNS LEEIHIFVLC NILRRPIIVI SDKMLRSLES GSNFAPLKVG GIYLPLHWPA |
| 241 | QECYRYPIVL GYDSHHFVPL VTLKDSGPEI RAVPLVNRDR GRFEDLKVHF LTDPENEMKE |
| 301 | KLLKEYLMVI EIPVQGWDHG TTHLINAAKL DEANLPKEIN LVDDYFELVQ HEYKKWQENS |
| 361 | EQGRREGHAQ NPMEPSVPQL SLMDVKCETP NCPFFMSVNT QPLCHECSER RQKNQNKLPK |
| 421 | LNSKPGPEGL PGMALGASRG EAYEPLAWNP EESTGGPHSA PPTAPSPFLF SETTAMKCRS |
| 481 | PGCPFTLNVQ HNGFCERCHN ARQLHASHAP DHTRHLDPGK CQACLQDVTR TFNGICSTCF |
| 541 | KRTTAEASSS LSTSLPPSCH QRSKSDPSRL VRSPSPHSCH RAGNDAPAGC LSQAARTPGD |
| 601 | RTGTSKCRKA GCVYFGTPEN KGFCTLCFIE YRENKHFAAA SGKVSPTASR FQNTIPCLGR |
| 661 | ECGTLGSTMF EGYCQKCFIE AQNQRFHEAK RTEEQLRSSQ RRDVPRTTQS TSRPKCARAS |
| 721 | CKNILAGRSE ELCMECQHPN QRMGPGAHRG EPAPEDPPKQ RCRAPACDHF GNAKCNGYCN |
| 781 | ECFQFKQMYG |

Such immunoassays can involve the use of an agent (e.g., an antibody) specific to TNFAIP3. An agent such as an antibody that “specifically binds” to TNFAIP3 is a term well understood in the art, and methods to determine such specific binding are also well known in the art. An antibody is said to exhibit “specific binding” if it reacts or associates more frequently, more rapidly, with greater duration and/or with greater affinity with TNFAIP3 than it does with other proteins. It is also understood by reading this definition that, for example, an antibody that specifically binds to a first target peptide may or may not specifically or preferentially bind to a second target peptide. As such, “specific binding” or “preferential binding” does not necessarily require (although it can include) exclusive binding. Generally, but not necessarily, reference to binding means preferential binding. In some examples, an antibody that “specifically binds” to a target peptide or an epitope thereof may not bind to other peptides or other epitopes in the same antigen.

As used herein, the term “antibody” refers to a protein that includes at least one immunoglobulin variable domain or immunoglobulin variable domain sequence. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J Immunol. 1996; 26(3):629-39)) as well as complete antibodies. An antibody can have the structural features of IgA, IgG, IgE, IgD, IgM (as well as subtypes thereof). Antibodies may be from any source including, but not limited to, primate (human and non-human primate) and primatized (such as humanized) antibodies.

A number of antibodies that bind to TNFAIP3 are commercially available, e.g., from Abbexa Ltd; Abcam; Abeomics; Abnova Corporation; Affinity Biosciences; Bethyl Laboratories, Inc.; Biorbyt; Bioss Inc.; BosterBio; Cell Signaling Technology; Creative Biolabs; Creative Diagnostics; CUSABIO Technology LLC; Elabscience Biotechnology Inc.; Enzo Life Sciences, Inc.; EpiGentek; FabGennix International, Inc.; G Biosciences; GeneTex; HUABIO; Leading Biology; LifeSpan BioSciences; MilliporeSigma; MyBioSource.com; Novus Biologicals; NSJ Bioreagents; OriGene Technologies; ProSci, Inc; Proteintech Group Inc; R&D Systems; RayBiotech; Santa Cruz Biotechnology, Inc.; Sino Biological, Inc.; St John's Laboratory; Thermo Fisher Scientific; United States Biological; and Wuhan Fine Biotech Co., Ltd.

In some embodiments, the antibodies as described herein can be conjugated to a detectable label and the binding of the detection reagent to TNFAIP3 can be determined based on the intensity of the signal released from the detectable label. Alternatively, a secondary antibody specific to the detection reagent can be used. One or more antibodies may be coupled to a detectable label. Any suitable label known in the art can be used in the assay methods described herein. In some embodiments, a detectable label comprises a fluorophore. As used herein, the term “fluorophore” (also referred to as “fluorescent label” or “fluorescent dye”) refers to moieties that absorb light energy at a defined excitation wavelength and emit light energy at a different wavelength. In some embodiments, a detection moiety is or comprises an enzyme. In some embodiments, an enzyme is one (e.g., β-galactosidase) that produces a colored product from a colorless substrate.

In some examples, an assay method described herein is applied to measure the level of TNFAIP3 in a sample, which can be a blood sample or a serum sample. Any of the assays known in the art, e.g., immunoassays can be used for measuring the level of TNFAIP3.

It will be apparent to those of skill in the art that this disclosure is not limited to immunoassays. Detection assays that are not based on an antibody, such as mass spectrometry, are also useful for the detection and/or quantification of TNFAIP3. Assays that rely on a chromogenic substrate can also be useful for the detection and/or quantification of TNFAIP3.

Alternatively, the level of nucleic acids encoding TNFAIP3 in a sample can be measured, e.g., via a conventional method. In some embodiments, measuring the expression level of nucleic acid encoding TNFAIP3 comprises measuring mRNA. In some embodiments, the expression level of mRNA encoding TNFAIP3 can be measured using real-time reverse transcriptase (RT) Q-PCR or a nucleic acid microarray. Methods to detect biomarker nucleic acid sequences include, but are not limited to, polymerase chain reaction (PCR), reverse transcriptase-PCR (RT-PCR), in situ PCR, quantitative PCR (Q-PCR), real-time quantitative PCR (RT Q-PCR), in situ hybridization, Southern blot, Northern blot, sequence analysis, microarray analysis, detection of a reporter gene, or other DNA/RNA hybridization platforms.

An exemplary sequence of human TNFAIP3 nucleic acid is provided in GenBank as Acc. No. NM_001270508.2 (variant 1); NM_001270507.2 (variant 2); or NM_006290.4 (variant 3) (all three variants encode the same protein), all of which are incorporated herein by reference.

Any binding agent that specifically binds to TNFAIP3 may be used in methods described herein to measure the level of TNFAIP3 in a sample. In some embodiments, the binding agent is an antibody or an aptamer that specifically binds to TNFAIP3 protein. In other embodiments, the binding agent may be one or more oligonucleotides complementary to TNFAIP3 nucleic acid.

To measure the level of a target biomarker, a sample can be in contact with a binding agent under suitable conditions. In general, the term “contact” refers to an exposure of the binding agent with the sample or cells collected therefrom for suitable period sufficient for the formation of complexes between the binding agent and TNFAIP3 (e.g., nucleic acid or protein) in the sample, if any. In some embodiments, the contacting is performed by capillary action in which a sample is moved across a surf ace of the support membrane.

In some embodiments, the assays can be performed on low-throughput platforms, including single assay format. For example, a low-throughput platform can be used to measure the presence and amount of TNFAIP3 protein in a sample (e.g., a serum sample) for diagnostic methods, monitoring of disease and/or treatment progression, and/or predicting whether a disease or disorder may benefit from a particular treatment.

In some embodiments, it may be necessary to immobilize a binding agent to the support member. Methods for immobilizing a binding agent will depend on factors such as the nature of the binding agent and the material of the support member and may require particular buffers. Such methods will be evident to one of ordinary skill in the art. For example, TNFAIP3 in a sample can be measured using any method described herein.

The type of detection assay used for the detection and/or quantification of TNFAIP3 may depend on the particular situation in which the assay is to be used (e.g., clinical or research applications), on the kind of TNFAIP3 to be detected (e.g., nucleic acid or protein), and/or on the kind and number of patient samples to be run in parallel, to name a few parameters.

The assay methods described herein may be used for both clinical and non-clinical purposes. Some examples are provided herein.

### II. Application of Detection of TNFAIP3

Methods described herein can be applied for evaluation of MOG-AAD, e.g., diagnosis, prognosis, or relapse of MOG-AAD. Evaluation can include identifying a subject as being at risk for or having MOG-AAD or a relapse thereof. Evaluation can also include monitoring treatment of a disease, such as evaluating the effectiveness of a treatment for MOG-AAD.

(a) Diagnosis, Prognosis, or Relapse

In some embodiments, the methods described herein are used to determine the level of TNFAIP3 in a sample (e.g., a serum sample) collected from a subject (e.g., a human patient suspected of having MOG-AAD or a relapse thereof). The TNFAIP3 level is then compared to a reference value to determine whether the subject has or is at risk for MOG-AAD or a relapse thereof. The reference value can be a control level of TNFAIP3. In some embodiments, the control level is a level of TNFAIP3 in a control sample. In some embodiments, a control sample is obtained from a healthy subject or population of healthy subjects. As used herein, a healthy subject is a subject that is apparently free of MOG-AAD at the time the level of TNFAIP3 is measured or has no history of MOG-AAD. In some embodiments, a control sample is obtained from a subject with MOG-AAD who is in remission or population of subjects with MOG-AAD who are in remission.

In some embodiments, the amount by which the level (or score) in the subject is less than the reference level (or score) is sufficient to distinguish a subject from a control subject, and optionally is a statistically significantly less than the level (or score) in a control subject. In cases where the level (or score) of TNFAIP3 in a subject being equal to the reference level (or score) of TNFAIP3, the “being equal” refers to being approximately equal (e.g., not statistically different).

Suitable reference values can be determined using methods known in the art, e.g., using standard clinical trial methodology and statistical analysis. The reference values can have any relevant form. In some cases, the reference comprises a predetermined value for a meaningful score or level of TNFAIP3, e.g., a control reference level that represents a normal level of TNFAIP3, e.g., a level in an unaffected subject or a subject who is not at risk of developing MOG-AAD or a subject with MOG-AAD in remission, and/or a disease reference that represents a level of TNFAIP3 associated with MOG-AAD or a relapse thereof.

The predetermined level or score can be a single cut-off (threshold) value, such as a median or mean, or a level or score that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level or score is a level or score determined in the same subject, e.g., at a different time point, e.g., an earlier time point.

The control level can also be a predetermined level. Such a predetermined level can represent the level of TNFAIP3 in a population of subjects that do not have or are not at risk for MOG-AAD or a population of subject with MOG-AAD who are in remission. The predetermined level can take a variety of forms. For example, it can be a single cut-off value, such as a median or mean. In some embodiments, such a predetermined level can be established based upon comparative groups, such as where one defined group is known to have MOG-AAD and another defined group is known to not have MOG-AAD. Alternatively, the predetermined level can be a range, for example, a range representing the levels of TNFAIP3 in a control population within a predetermined percentile.

The control level as described herein can be determined by various methods. In some embodiments, the control level can be obtained by performing a known method. In some embodiments, the control level can be obtained by performing the same assay used for determine the level of TNFAIP3 in a sample from a subject. In some embodiments, the control level can be obtained by performing a method described herein. In some embodiments, the control level can be obtained from members of a control population and the results can be analyzed by, e.g., a computational program, to obtain the control level (a predetermined level) that represents the level of TNFAIP3 in the control population.

By comparing the level of TNFAIP3 in a sample obtained from a subject to the reference value as described herein, it can be determined as to whether the subject has or is at risk for MOG-AAD or a relapse thereof. For example, if the level of TNFAIP3 of the subject is decreased from the reference value, the candidate subject might be identified as having or at risk for MOG-AAD or a relapse thereof.

As used herein, “a decreased level or a level below a reference value” means that the level of TNFAIP3 is lower than a reference value, such as a predetermined threshold or a level of TNFAIP3 in a control sample.

An decreased level of TNFAIP3 includes a TNFAIP3level that is, for example, 1%, 5%, 10%, 20%, 30%, 40%, 50%, 60%, 70%, 80%, 90%, 100%, 150%, 200%, 300%, 400%, 500% or more below a reference value. A decreased level of TNFAIP3 also includes decreasing a phenomenon from a non-zero state (e.g., some or detectable TNFAIP3 in a sample) to a zero state (e.g., no or undetectable TNFAIP3 in a sample).

As used herein, “an elevated level or a level above a reference value” means that the level of TNFAIP3 is higher than a reference value, such as a predetermined threshold or a level of TNFAIP3 in a control sample.

An elevated level of TNFAIP3 includes a TNFAIP3 level that is, for example, 1%, 5%, 10%, 20%, 30%, 40%, 50%, 60%, 70%, 80%, 90%, 100%, 150%, 200%, 300%, 400%, 500% or more above a reference value. An elevated level of TNFAIP3 also includes increasing a phenomenon from a zero state (e.g., no or undetectable TNFAIP3 in a sample) to a non-zero state (e.g., some or detectable TNFAIP3 in a sample).

In some embodiments, the subject is a human patient having a symptom of MOG-AAD, e.g., those disclosed herein such as ADEM, CIS, ON, recurrent forms of ADEM and ON, TM, and NMO-SD. For example, the subject has loss or blurring of vision in one or both eyes, loss of color vision, paralysis of a limb or limbs, paraparesis of a limb or limbs, loss of sensation, loss of bladder or bowel control, profound bladder retention, seizures, or a combination thereof. In some embodiments, the subject has one or more symptoms of MOG-AAD, has a history of one or more symptoms of MOG-AAD, or a history of MOG-AAD at the time the sample is collected. In some embodiments, the subject has no symptom of MOG-AAD, has no history of a symptom of MOG-AAD, or no history of MOG-AAD at the time the sample is collected. In some embodiments, the subject is having a relapse of MOG-AAD.

(b) Evaluation of Treatment Effectiveness

Methods described herein can also be applied to evaluate the effectiveness of a treatment for MOG-AAD. For example, multiple samples (e.g., serum samples) can be collected from a subject to whom a treatment is performed either before and after the treatment or during the course of the treatment. The levels of TNFAIP3 can be measured by any method described herein. If the level of TNFAIP3 increases after the treatment or over the course of the treatment (the level of TNFAIP3 in a later collected sample as compared to that in an earlier collected sample), remains the same or increases, it indicates that the treatment is effective.

In the subject is identified as not responsive to the treatment, a higher dose and/or frequency of dosage of the therapy can be administered to the subject identified. In some embodiments, the dosage or frequency of dosage of the therapy is maintained, lowered, or ceased in a subject identified as responsive to the treatment or not in need of further treatment. Alternatively, a different treatment can be applied to the subject who is found as not responsive to the first treatment.

(c) Non-Clinical Applications

Methods described herein can also be applied to non-clinical uses, e.g., for research purposes. For example, methods described herein can be used to identify novel biological pathways or processes involved in MOG-AAD.

In some embodiments, methods described herein can be applied to the development of a new therapy. For example, the levels of TNFAIP3 can be measured in samples obtained from a subject having been administered a new therapy (e.g., in a clinical trial). In some embodiments, the level of TNFAIP3 can indicate the efficacy of the new therapy or the progress of MOG-AAD in the subject prior to, during, or after the new therapy.

### III. Treatment of MOG-AAD

A subject having or at risk for MOG-AAD, as identified using the methods described herein, may be treated with any appropriate therapy. Non-limiting examples of a therapy for use in methods described herein include an immunosuppressive agent (e.g., mycophenolate mofetil, azathioprine, rituximab, prednisone), plasma exchange (PLEX) therapy, intravenous immunoglobulin (IVIG), or TNFAIP3 (e.g., TNFAIP3 protein, nucleic acids encoding TNFAIP3 protein, or an agent that increases expression of TNFAIP3).

In some embodiments, methods provided herein include selecting a treatment for a subject based on the output of the described method, e.g., measuring the level of TNFAIP3. Alternatively, or in addition to, methods provided herein include administering a treatment for a subject based on the output of the described method.

In some embodiments, the therapy comprises administering an immunosuppressive agent such as mycophenolate mofetil, azathioprine, or rituximab. In some embodiments, the therapy comprises administering a steroid such as prednisone. In some embodiments, the therapy comprises performing plasma exchange (PLEX) therapy. In some embodiments, the therapy comprises administering intravenous immunoglobulin (IVIG). In some embodiments, the therapy comprises administering TNFAIP3 such as TNFAIP3 protein, nucleic acids encoding TNFAIP3 protein, or an agent that increases expression of TNFAIP3.

An effective amount of the therapy can be administered to a subject (e.g., a human) in need of the treatment via any suitable route, such as intravenous administration, e.g., as a bolus or by continuous infusion over a period of time, by intramuscular, intraperitoneal, intracerebrospinal, subcutaneous, intra-articular, intrasynovial, intrathecal, oral inhalation, or topical routes.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or virtually any other reason.

Empirical considerations such as the half-life of an agent will generally contribute to the determination of the dosage. Frequency of administration can be determined and adjusted over the course of therapy, and is generally, but not necessarily, based on treatment and/or suppression and/or amelioration and/or delay of MOG-AAG. Alternatively, sustained continuous release formulations of therapeutic agent may be appropriate. Various formulations and devices for achieving sustained release are known in the art.

As used herein, the term “treating” refers to the application or administration of a composition including one or more active agents to a subject who has MOG-AAG, a symptom of MOG-AAG, and/or a predisposition toward MOG-AAG, with the purpose to cure, heal, alleviate, relieve, alter, remedy, ameliorate, improve, or affect the disorder, the symptom of the MOG-AAG, and/or the predisposition toward MOG-AAG.

Alleviating MOG-AAG includes delaying the development or progression of the disease, and/or reducing disease severity. Alleviating the disease does not necessarily require curative results.

As used herein, “delaying” the development of a disease (e.g., MOG-AAG) means to defer, hinder, slow, retard, stabilize, and/or postpone progression of the disease. This delay can be of varying lengths of time, depending on the history of the disease and/or individuals being treated. A method that “delays” or alleviates the development of a disease and/or delays the onset of the disease is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques known in the art. However, development also refers to progression that may be undetectable. For purposes of this disclosure, development or progression refers to the biological course of the symptoms. “Development” includes occurrence, recurrence, and onset. As used herein, “onset” or “occurrence of MOG-AAG includes initial onset and/or recurrence. As used herein, “relapse” of MOG-AAG refers to worsening or recurrence of MOG-AAG or the signs and symptoms of MOG-AAG after a period of complete or partial recovery or remission.

In some embodiments, the therapy is administered one or more times to the subject. In some embodiments, the therapy comprises two or more types of therapies that can be administered as part of a combination therapy for treatment of MOG-AAG (e.g., a combination therapy comprising an immunosuppressive agent and plasma exchange (PLEX) therapy).

The term combination therapy, as used herein, embraces administration of these agents in a sequential manner, that is wherein each therapeutic agent is administered at a different time, as well as administration of these therapeutic agents, or at least two of the agents, in a substantially simultaneous manner.

Sequential or substantially simultaneous administration of each agent can be affected by any appropriate route including, but not limited to, oral routes, intravenous routes, intramuscular routes, subcutaneous routes, and direct absorption through mucous membrane tissues. The agents can be administered by the same route or by different routes. For example, a first agent can be administered orally, and a second agent can be administered intravenously.

As used herein, the term “sequential” means, unless otherwise specified, characterized by a regular sequence or order, e.g., if a dosage regimen includes the administration of a first therapeutic agent and a second therapeutic agent, a sequential dosage regimen could include administration of the first therapeutic agent, before, simultaneously, substantially simultaneously, or after administration of the second therapeutic agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents described herein.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

In order that the invention described may be more fully understood, the following examples are set forth. The examples described in this application are offered to illustrate the methods and compositions provided herein and are not to be construed in any ways as limiting their scope.

### Materials and Methods

The following materials and methods were used in the Examples set forth herein.

### Subjects and Blood Samples

All methods in this study were carried out in accordance with relevant guidelines and regulations. Subjects were selected from an ongoing biomarker study at the Partners Pediatric MS Center at Massachusetts General Hospital, which is approved by Partners Human Research Committee/Institutional Review Board for the use of human material. Parents of children signed an informed consent form. Peripheral venous blood was collected in lithium heparin blood collection tubes (Becton Dickinson, NJ, USA) from subjects after obtaining informed consent. We included 20 pediatric MOG-AAD patients and 44 age and sex-matched pediatric healthy controls (PHCs). MOG antibody testing was performed at the sample collection site by cell-based assay (Fernandez-Carbonell et al. Clinical and MRI phenotype of children with MOG antibodies. Mult. Scler. 22, 174-184 (2016)), or at the Mayo Clinic as part of clinical care. The 20 MOG-AAD patients had the following diagnoses at the time of sample collection according to International Pediatric MS Study Group diagnostic criteria: 7 with MS, 7 with ADEM-ON, 1 with multiphasic ADEM, 1 with ADEM-TM, 1 with CIS, 1 with a demyelinating neurological disorder and 2 with NMO-SD48. Patients were diagnosed with NMO-SD if presenting with ON, TM and at least two of these 3 criteria: MRI evidence of a continuous spinal cord lesion, brain MRI that was non-diagnostic of MS, and NMO IgG seroposivity (Krupp et al. International Pediatric Multiple Sclerosis Study Group criteria for pediatric multiple sclerosis and immunemediated central nervous system demyelinating disorders: revisions to the 2007 definitions. Mult. Scler. 19, 1261-1267 (2013); Gombolay & Chitnis Pediatric neuromyelitis optica spectrum disorders. Curr. Treat. Opt. Neurol. 20, 19 (2018)). Out of the 20 MOG-AAD patients, 15 patients had longitudinal samples, including treated/untreated and relapse/non-relapse samples. Untreated samples were defined as no steroids or intravenous immunoglobulin nor disease modifying therapies within 30 days prior to sample collection. Samples within 30 days of a clinical relapse with new MRI lesions were defined as “relapse” samples or within 64 days of ongoing relapse symptoms, while samples at a non-relapse time point were defined as remission samples. One sample (#1.3) was identified as a pre-relapse sample, since the patient reported new mild clinical symptoms, but no radiological correlation was found (Table 1). A second sample (#5.5) was also termed pre-relapse since the patient had significant new headache not responsive to standard medication. Neurologists specialized in pediatric demyelinating disorders validated all clinical and radiological data (TC and GG).

TABLE 1
Demographics of MOG-AAD subjects and controls.

| | | Age range | |
|---|---|---|---|
| Subjects | Number | (years) | Sex ratio F:M |
| MOG-antibody associated disease | 24 | 4.5-52 | 15:9 |
| Pediatric healthy controls | 44 | 4-18 | 26:18 |

### Cell Stimulation Assay and FACS Analysis

Peripheral blood mononuclear cells (PBMCs) were isolated by Ficoll-Paque Plus (GE healthcare Biosciences AB, Sweden) density gradient centrifugation. Cells were cultured using Hybridomas and Lymphoid cells (HL-1, Lonza, MD, USA) media containing 5% of human serum (Valley Biomedical INC, VA, USA), L-Glutamine (Fisher Scientific, NH, USA), Penicillin-Streptomycin (Fisher Scientific, NH, USA), HEPES, non-essential amino acids (NEAA) and sodium pyruvate (all 3 from Lonza, MD, USA). PBMCs from 7 MOG-AAD patients of which 3 patients had longitudinal samples and 7 age and sex matched PHCs (Table 1) were plated at a density of 500,000 cells/well in a 96-well plate (Corning, ME, USA). Cells were stimulated by either one of the following 6 peptide conditions, (1) MOG p1-20, (2) MOG p35-55 (Immune Tolerance Network, USA), (3) MOG p119-130 (4) MOG p181-195 (5) MOG p186-200 (Genemed INC, CA, USA), (6) cocktail of myelin peptides consisting of Proteolipid Protein (PLP) 139-154, Myelin Basic Protein (MBP) 13-32, MBP 111-129 and MBP 146-170 (Immune Tolerance Network, USA) at 10 μg/ml. Cells were cultured for 7 days at 37° C., 5% CO₂ and 90% humidity.

To evaluate cytokine production, cells were further stimulated with Phorbol 12-myristate 13-acetate (PMA) at 33 ng/ml, Ionomycin at 166 ng/ml (Sigma Aldrich, MO, USA) and Golgi stop (BD Bioscience, CA, USA) for 4 h. After incubation cells were stained with the following antibodies, human anti-CD4 APC (RPA-T4, Biolegend Inc, CA, USA), anti-CD45RA-AF700 (HI100, Biolegend Inc, CA, USA), anti-CCR7-PE (GO43H7, Biolegend Inc, CA, USA) and live/dead fixable violet dead cell stain kit (Thermo Fischer Scientific, USA). Cells were then fixed and permeabilized with BD fixation and permeabilization buffer (BD Biosciences, CA, USA) and stained with the following intracellular cytokine antibodies: anti-IL17-PeCy7 (BL168, Biolegend Inc, CA, USA) and anti-IFNγ-PerCp (4S.B3, Biolegend Inc, CA, USA). Flow cytometry was performed on BD LSR II (BD Biosciences, CA, USA); data was analyzed using Flowjo vX.0.7 and the graphs were made using GraphPadPrism version 8.4.2 (464).

Live CD4+ T cells were selected from all lymphocytes and analyzed for expression of central memory cells (CMCs: CCR7+CD45RA−), effector memory cells (EMCs: CCR7-CD45RA−), and effector cells (CD45RA+). Intracellular pro-inflammatory cytokines, IL17+, IFNγ+, and IL17+IFNγ+(double positive) were gated within CMCs and EMCs.

### Gene Expression Assays and Data Analysis

Single cell RNA sequencing (inDrop). Single cell RNA sequencing was conducted in CMCs from CD4+ T cells isolated from MOG-AAD patient #1 with 3 untreated longitudinal samples, (1) at remission (MOG-AAD #1.2) (2) at a pre-relapse (MOG-AAD #1.3), two months prior to a relapse in which the patient experienced headache and mild visual symptoms with no radiological correlate and (3) at a relapse (MOG-AAD #1.4) characterized by bilateral weakness and multiple new MRI lesions (Table 1) following the inDrop technique as previously described (Altonsy et al. Context-dependent cooperation between nuclear factor kappaB (NF-kappaB) and the glucocorticoid receptor at a TNFAIP3 intronic enhancer: a mechanism to maintain negative feedback control of inflammation. J. Biol. Chem. 289, 8231-8239 (2014); Coornaert et al. T cell antigen receptor stimulation induces MALT1 paracaspase-mediated cleavage of the NF-kappaB inhibitor A20. Nat. Immunol. 9, 263-271 (2008)).

InDrop single cell library sequencing was performed at the Single Cell Core (SCC), laboratory of Dr. Allon Klein, Department of Systems Biology at Harvard Medical School (HMS). The relapse sample failed sequencing, however the remission and pre-relapse samples were evaluable. For simplicity, moving forward we reassigned the pre-relapse sample to be a relapse sample.

Whole PBMCs were stimulated with anti-CD3 (OKT3, Ebioscience, CA, USA) and anti-CD28 (CD28.2, Ebioscience, CA, USA) at 0.5 μg/ml for 3 days. Cells were stained with human anti-CD4 APC (RPA-T4, Biolegend Inc, CA, USA), anti-CD45RA-AF700 (HI100, Biolegend Inc, CA, USA), anti-CCR7-PE (GO43H7, Biolegend Inc, CA, USA) and live/dead fixable violet dead cell stain kit (Thermo Fischer Scientific, USA). CMCs (CCR7+CD45RA−) were sorted from CD4+ T cells using BD FACS ARIA (BD Biosciences, CA, USA). 3,000 cells from a cell suspension comprising of CMCs from the 2 samples were isolated into droplets that contained lysis buffer. cDNA libraries were sequenced using the Illumina NextSeq 500 platform and analyzed following V3 Indrop criteria. After sequencing the raw BCL files were manually demultiplexed using the bcl2fastq software by illumina. Reads obtained from bcl2fastq were further processed using the single-cell RNA-seq pipeline of the bcbio-nextgen software suite. The single-cell RNA-seq pipeline inspected each read, performed alignment using RapMap (Srivastava et al. RapMap: a rapid, sensitive and accurate tool for mapping RNA-seq reads to transcriptomes. Bioinformatics 32, i192-i200 (2016)) and produced transcript level count matrix. This matrix was further processed with Seurat where QC, filtering, log normalization and scaling was performed. The scaled data was further clustered using Seurat and visualized using TSNE (van der Maaten & Hinton Visualizing data using t-SNE. J. Mach. Learn. Res. 9, 2579-2605 (2008)).

The TSNE plot was labeled with sample state (Relapse/Remission) to identify cluster differences between states. Since the clusters showed homogeneity between relapse and remission sample states, gene specific differences between the samples were assessed. “AverageExpression” function in Seurat was used to calculate average gene expression for the relapse and remission samples separately. The difference in the gene expression was calculated by subtracting the expression values from relapse and remission samples for each gene. The obtained list was further sorted to identify genes that were up regulated in relapse and remission samples respectively. Based on the difference in the gene expression, the top 5 genes were further evaluated.

Digital gene expression (DGE) sequencing. DGE sequencing was conducted in CMCs from CD4+ T cells isolated from the same MOG-AAD patient #1 with 3 untreated longitudinal samples at remission (MOG-AAD #1.2), pre-relapse (MOG-AAD #1.3) and relapse (MOG-AAD #1.4) as previously described (Table 1) (Lu et al. Dimerization and ubiquitin mediated recruitment of A20, a complex deubiquitinating enzyme. Immunity 38, 896-905 (2013); Lee et al. Failure to regulate TNF-induced NF-kappaB and cell death responses in A20-deficient mice. Science 289, 2350-2354 (2000); Onizawa, M. et al. The ubiquitin-modifying enzyme A20 restricts ubiquitination of the kinase RIPK3 and protects cells from necroptosis. Nat. Immunol. 16, 618-627 (2015)). PBMCs were stimulated with MOG peptides, p35-55, p119-130 and p181-195 at 10 μg/ml for 7 days. Cells were stained with human anti-CD4 APC (RPA-T4, Biolegend Inc, CA, USA), anti-CD45RA-AF700 (HI100, Biolegend Inc, CA, USA), anti-CCR7-PE (GO43H7, Biolegend Inc, CA, USA) and live/dead fixable violet dead cell stain kit (Thermo Fischer Scientific, USA). CMCs (CCR7+CD45RA−) were sorted from CD4+ T cells using BD FACS ARIA. Total RNA was isolated using Total RNA purification kit following manufacturer's guidelines (Norgen, MA, USA). Based on the guidelines provided by the Broad institute for library preparation, RNA concentration for all samples was normalized to 5 ng/μl 1 in 10 μl of nuclease free water. Library sequencing was performed at The Broad Genomics Platform.

Raw BCL files generated through sequencing were further de-multiplexed using Picard and the resulting FASTQ files where aligned to the human reference genome (GRCh38) using the STAR v2.4.2a (Dobin et al. STAR: ultrafast universal RNA-seq aligner. Bioinformatics 29, 15-21 (2013)) aligner. Further QC was done using the RNA-seQC (DeLuca et al. RNA-SeQC: RNA-seq metrics for quality control and process optimization. Bioinformatics 28, 1530-1532 (2012)) and transcript counts were produced using feature Counts function of the Subread package (Liao et al. featureCounts: an efficient general purpose program for assigning sequence reads to genomic features. Bioinformatics 30, 923-930 (2014)). Before running the analysis, genes with low overall expression were removed from the analysis and the data was normalized using the DESeq2 package (Love et al. Moderated estimation of fold change and dispersion for RNA-seq data with DESeq2. Genome Biol. 15, 550 (2014)). The graphs were made using GraphPadPrism version 8.4.2 (464).

Nanostring ncounter gene expression assay. Cell subsets were isolated from MOG-AAD patient #2 with 3 longitudinal samples, 1 at relapse (MOG-AAD #2.1) and 2 mycophenolate mofetil treated samples at remission (MOG-AAD #2.2 and MOG-AAD #2.3) (Table 1) were evaluated by NanoString array as previously described (Urbano et al. TNF-alpha-induced protein 3 (TNFAIP3)/A20 acts as a master switch in TNF-alpha blockade-driven IL-17A expression. J. Allergy Clin. Immunol. 142, 517-529 (2018)). RNA expression of 770 genes was detected by nCounter XT Code-Set Gene Expression Assay, Human Autoimmune kit. CD4+ T cells, CD19+B cells and CD14+monocytes were positively selected using micro beads and magnetically isolated using MACSQuant columns placed in the magnetic field of a MACS separator (Miltenyi Biotec, CA, USA). In order to achieve maximum purity, staining antibodies for anti-CD4-PE (OKT4, Biolegend Inc, CA, USA) anti-CD19-PE (HIB19, Biolegend Inc, CA, USA) and anti-CD14-FITC (M5E2, BD Biosciences, CA, USA) were added during magnetic separation. 7-AAD viability staining solution was used to separate the live cells from the dead. Further CD4+, CD19+ and CD14+ cells were sorted using BD FACS ARIA (BD Biosciences, CA, USA). Total RNA was isolated using Total RNA purification kit following manufacturer's guidelines (Norgen, MA, USA). RNA concentration for all samples was normalized to 30 ng/μl in 5 μl of nuclease free water. Hybridization protocol for nCounter XT Code-Set Gene Expression Assay was performed following the manufacturer's instructions. Data were normalized and analyzed using nSolver software via the geometric mean of included housekeeping genes. The graphs were made using GraphPadPrism version 8.4.2 (464).

Quantification by real time PCR. CD4+ T cells from 7 MOG-AAD patients (longitudinal n=5/7, relapse n=7, remission n=8, total n=15, Table 1) were isolated as previously described. Total RNA was isolated using Total RNA purification kit following manufacturer's guidelines (Norgen, MA, USA). RNA concentration for all samples was determined using NanoDrop 2000/2000c Spectrophotometer (Thermo fischer scientific, USA). First-strand cDNA synthesis was performed for each RNA sample from 50 ng of total RNA using SuperScript IV VILO Master Mix (Thermo fischer scientific, USA) following manufacturer's guidelines. qPCR was performed using FAM-labeled primers for TNFAIP3, Hs00234713_m1 and GAPDH, Hs99999905_m1 with TaqMan Fast Universal qPCR Master Mix (No ampErase Uracil N-Glycosylase, Thermo fischer scientific, USA). Samples were run in duplicates on QuantStudio 7 Flex (Applied Biosystems, Life Technologies, USA). GAPDH gene was used as an endogenous control to normalize for differences in the amount of total RNA in each sample, and all values are shown as relative expression. The graphs were made using GraphPadPrism version 8.4.2 (464).

### SDS-PAGE and Western Blot

PBMCs from an untreated MOG-AAD patient #3 with 2 longitudinal samples at relapse (MOG-AAD #3.1) and non-relapse (remission, MOG-AAD #3.2) (Table 1) were cultured at a density of 250,000 cells/well in a 96-well plate (Corning, ME, USA) for 4, 8, 16 and 24 h under the following 2 conditions: (1) MOG antigen at 1 μg/ml (peptide cocktail comprising of MOG p1-20, p35-55, p119-130, p181-195 and p186-200, Sigma Aldrich, MO, USA), and (2) MOG antigen at 1 μg/ml with Dexamethasone at 100 nM (Sigma Aldrich, MO, USA). PBMCs for an additional MOG-AAD patient #2 with 2 longitudinal samples, untreated at relapse (MOG-AAD #2.1) and treated with mycophenolate mofetil at non-relapse (remission, MOG-AAD #2.3) (Table 1) were cultured similarly under the following 5 conditions: (1) Exvivo (unstimulated) (2) MOG antigen stimulation at a lower dose of 1 g/ml, (3) MOG antigen stimulation at a higher dose of 10 μg/ml (4) MOG antigen stimulation with dexamethasone at a lower dose of 1 μg+100 nM, and (5) MOG antigen stimulation with dexamethasone at a higher dose of 10 μg+1000 nM. Cells were lysed with RIPA lysis buffer (Thermo Fisher Scientific, MA, USA) supplemented with protease and phosphatase inhibitor cocktails (Thermo Fisher Scientific, MA, USA). Total protein was determined by Pierce BCA protein assay following manufacturer's instructions (Thermo Fisher Scientific, MA, USA). Samples were prepared with 30 μg of cell lysate, loading buffer (Life Technologies, OR, USA), and reducing agent (Life Technologies, OR, USA) and then heated for 10 min at 95° C. before use. Samples were run on 4-12% Bis Tris gels (Invitrogen, CA, USA) and transferred to polyvinylidene difluoride membranes (PVDF) (Immobilon-P, Millipore Sigma, MA, USA). Membranes were blocked using 5% BSA (Sigma Aldrich, MO, USA) and blotted overnight at 4° C. with antibodies against anti-TNFAIP3 (ab92324, 1:1,000), anti-NFκβ p50 (ab32360, 1:2000) Abcam, MA, USA, anti-phospho NFκβ p65 (3033 L, 1:2000), anti-NFκβ p65 (8242S, 1:2000), and anti-β-actin (4970S,1:4,000) Cell signaling, MA, USA. Membranes were incubated with secondary antibody anti-rabbit IgG HRP linked (7074P2, 1:10,000, Cell signaling, MA, USA) for 1 h at 37° C. Immunoblots were developed using KwikQuant western blot detection kit (Kindle biosciences, CT, USA). Protein bands were quantified using ImageJ version 1.53b and normalized to their respective β-actin. The graphs were made using GraphPadPrism version 8.4.2 (464).

### ELISA

TNFAIP3 concentration in serum samples of 50 MOG-AAD patients (Table 1) with relapse samples n=10, remission samples n=40 and PHC n=44 was assessed by commercially available TNFAIP3 ELISA kit (MyBiosource, CA, USA) following manufacturer's instructions. All samples were tested in duplicates. Detection range of the assay was 23.5 pg/ml-1500 pg/ml. The Intra-assay precision CV was <8% and Inter-assay precision CV was <10%. The optical density was determined at 450 nm and the reference wavelength was set at 560 nm. Nonlinear Mixed-Effects Models library in R (nlme) was used for statistical analysis. The graphs were made using GraphPadPrism version 8.4.2 (464). Multiplex panel comprising of 92 protein biomarkers (Olink Proteomics, Uppsala, Sweden) was used to assess serum samples of 49 MOG-AAD patients (Table 1) with relapse samples n=11 and remission samples n=38 following manufacturer's instructions. The Intra-assay precision CV was <15% and Inter-assay precision CV was <25%. Data from the analyzed protein biomarkers was presented in normalized protein expression (NPX) values, Olink Proteomics's arbitrary unit on log 2 scale (Assarsson et al. Homogenous 96-plex PEA immunoassay exhibiting high sensitivity, specificity, and excellent scalability. PLoS ONE 9, e95192 (2014); Lundberg et al. Homogeneous antibody-based proximity extension assays provide sensitive and specific detection of low-abundant proteins in human blood. Nucleic Acids Res. 39, e102 (2011)).

### Example 1: Flow Cytometric Analysis of MOG-Reactive CMC T Cells Show an Increased Percentage of IL17+, IFNγ+ and IL17+/IFNγ+ Cells

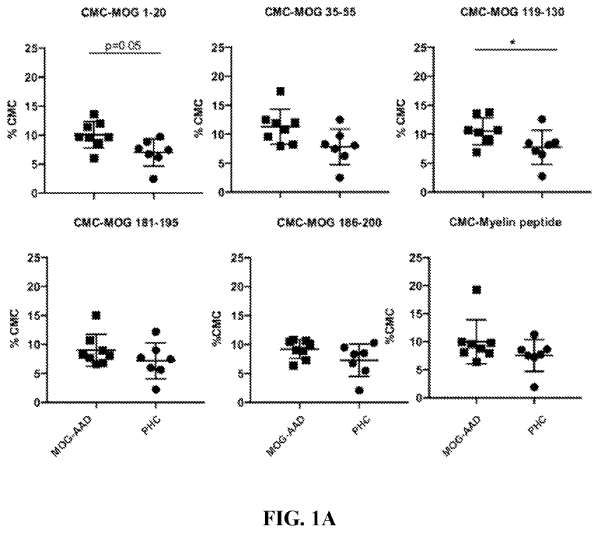

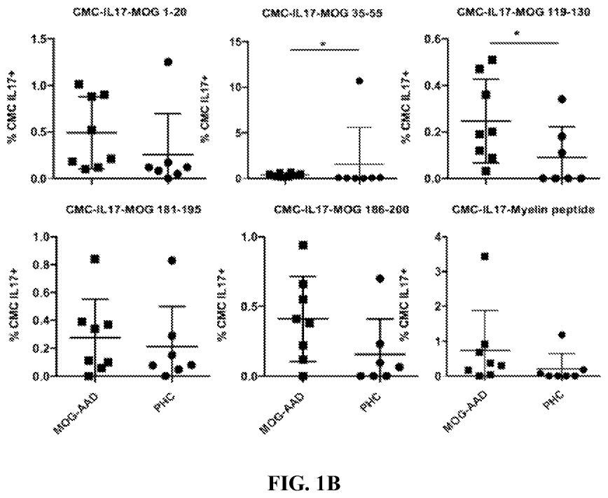

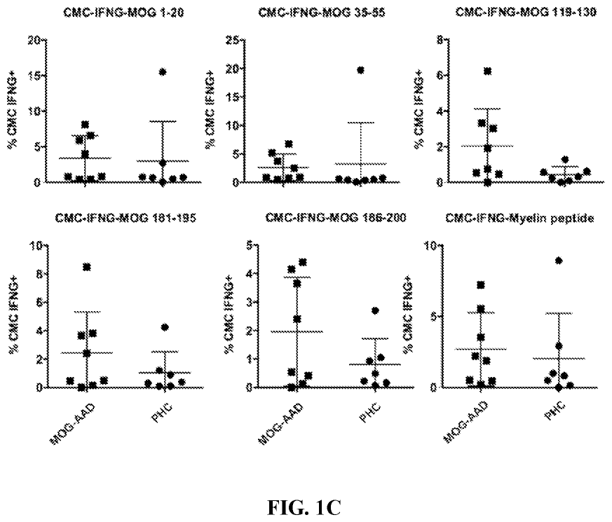

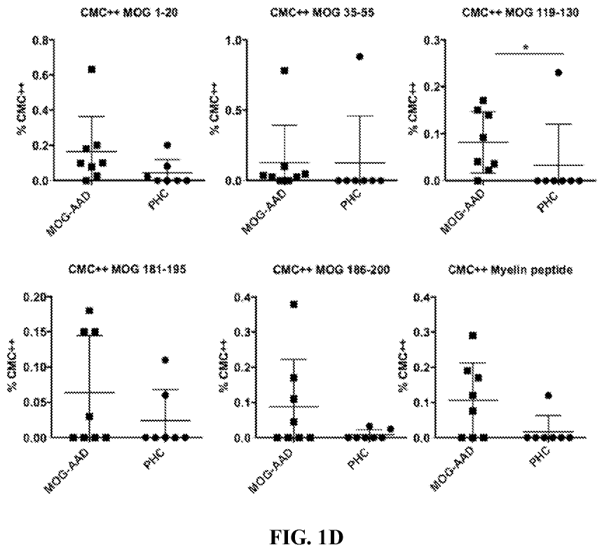

In this Example, the percentage of IL17+ and IFNγ+ cells in CMCs of untreated MOG-AAD patients (n=8) and age and sex matched PHCs (n=7) was evaluated (Table 1), when stimulated with peptides MOG p1-20, MOG p35-55, MOG p119-130, MOG p181-195, MOG p186-200 and myelin peptides. An increase in the percentage of CMCs, as well as IL17+ and IL17+/IFNγ+(double positive) CMCs in MOG-AAD patients when compared to PHCs was observed (FIGS. 1A-1D). (CMC-MOG 1-20, P=0.05, CMC-MOG 119-130, P=0.03; CMC-IL17+-MOG 35-55, P=0.02, CMCIL17+-MOG 119-130, P=0.03; CMC++ MOG 119-130, P=0.04).

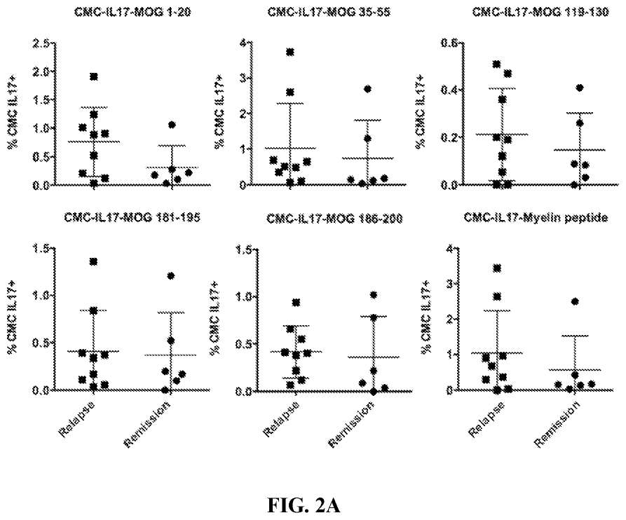

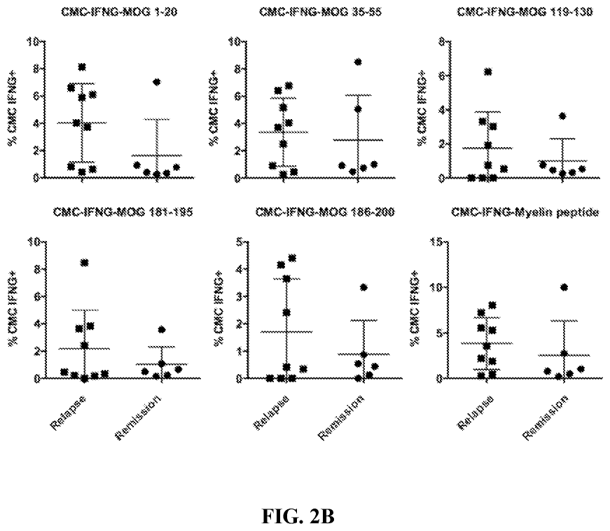

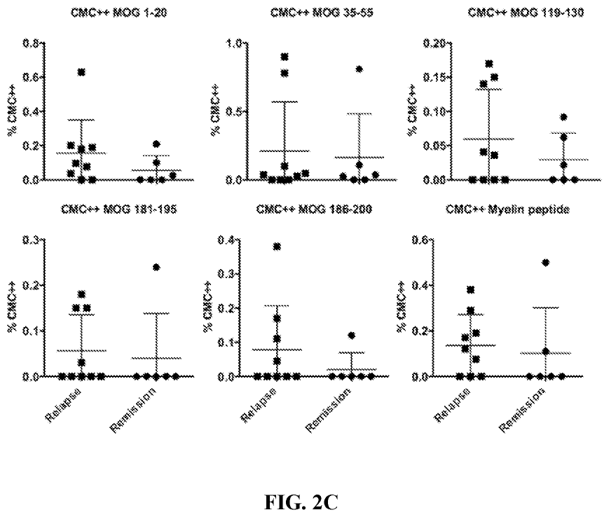

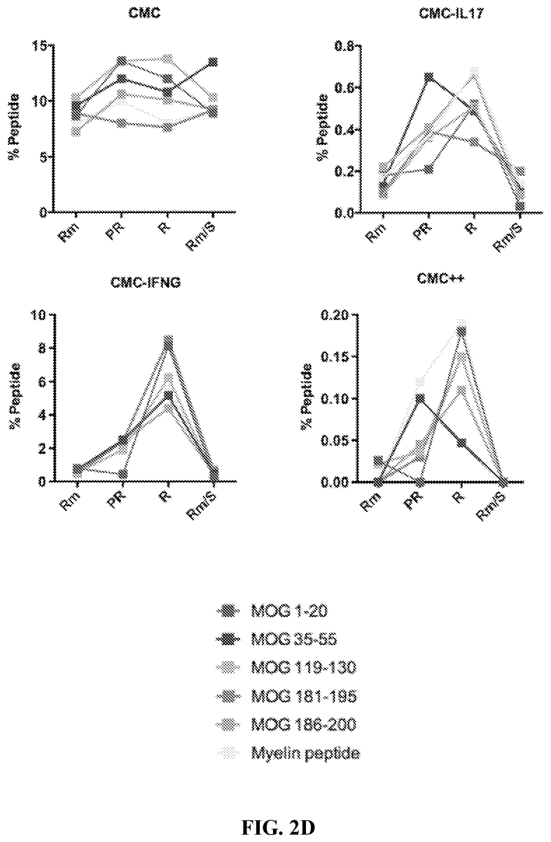

Next, a group of MOG-AAD patients with a blood sample within 30 days prior to a relapse (n=9) and during a remission time point (non-relapse, n=6) (Supplementary Table 1a) was identified. Interestingly, an increased proportion of IL17+, IFNγ+ and IL17+IFNγ+(double positive) CMCs after stimulation with several individual MOG peptides in MOG-AAD patients at the time of relapse as compared to remission time point was observed (FIG. 2A-2C).

Furthermore, PBMCs from MOG-AAD patient #1 with 4 longitudinal samples, (1) remission (MOGAAD #1.2) 2) pre-relapse (MOG-AAD #1.3) time point which was 2 months prior to a relapse during which the patient experienced headache and mild visual symptoms, (3) relapse (MOG-AAD #1.4) characterized by bilateral weakness and multiple new MRI lesions and (4) remission (MOG-AAD #1.5) on treatment with steroid were stimulated with individual MOG peptides. A dramatic increase in CMC IL17+ and CMC IFNγ+cells at a relapse was observed. Specifically, there was an increase in CMC IL17+cells responsive to MOG p35-55 in the 2 months prior to fulminant relapse, when the patient had mild visual symptoms (pre-relapse state) (FIG. 2D).

### Example 2: Gene Expression Analysis Revealed and Confirmed a Relapse Biomarker in MOG-AAD Patients

Since initial results demonstrated that CMC T cells might play a role in relapse and in multiphasic disease course in MOG-AAD patients, further evaluation of the transcriptomic profile of these cells was performed. Single cell RNA sequencing (inDrop) was performed on an untreated MOG-AAD patient #1 with 3 longitudinal samples during remission (MOG-AAD #1.2), pre-relapse (MOG-AAD #1.3) and at a relapse (MOGAAD #1.4). The relapse sample failed sequencing, however the pre-relapse and remission samples were evaluable. Given the evolving understanding that in MOG-AAD, clinical symptoms can occur prior to the presence of new MRI lesions (Sechi et al. Frequency and characteristics of MRI-negative myelitis associated with MOG autoantibodies. Mult Scler, 27(2): 303-308 (2021)), moving forward the pre-relapse sample was re-classified as relapse. Analysis was performed as described in “Materials and methods”.

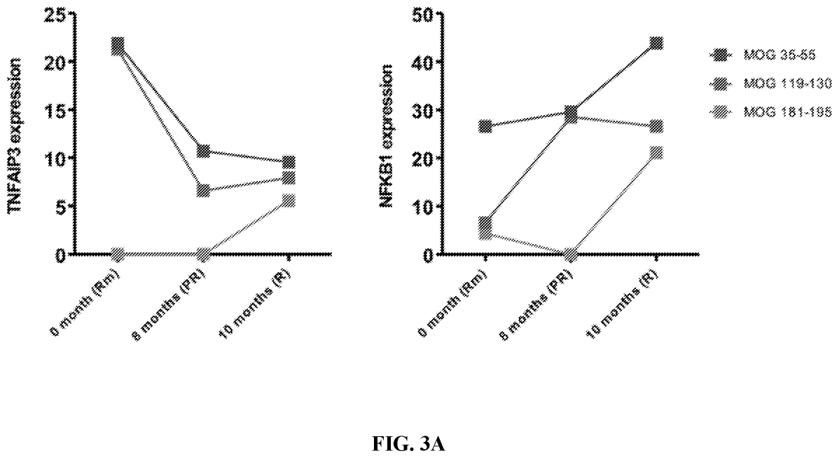

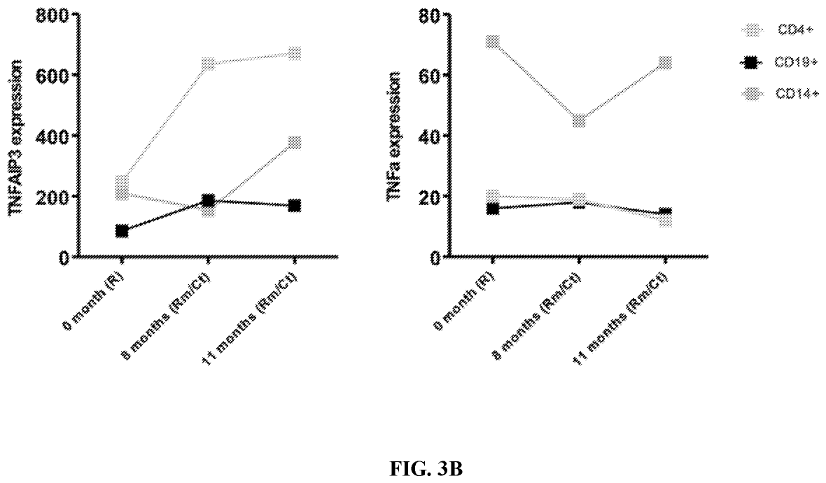

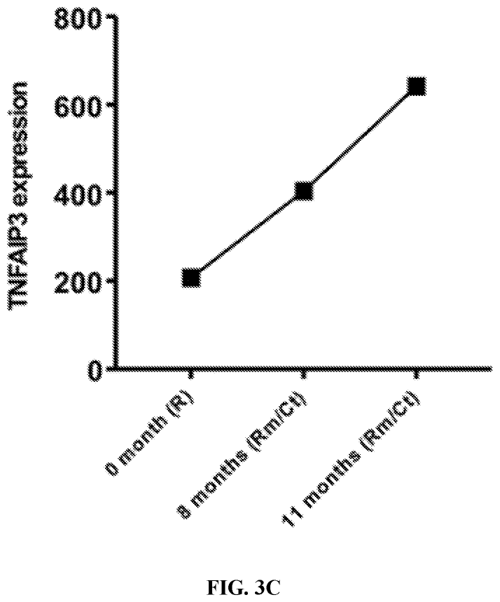

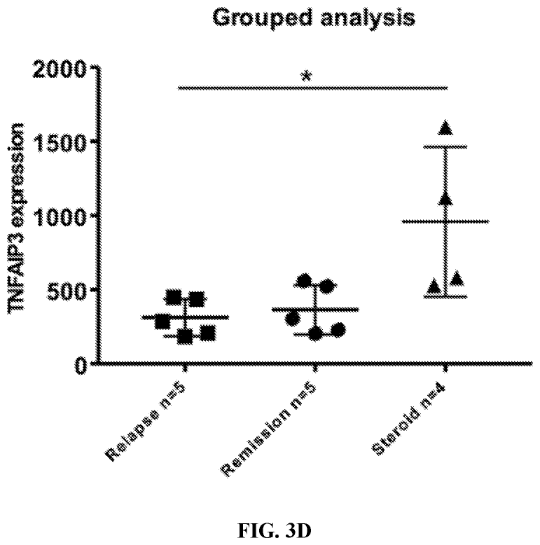

Cluster analysis of all the cells from relapse and remission samples using Seurat indicated that gene expression was homogeneous and there were no obvious differences between cells of relapse and remission samples (FIG. 3A). However, to identify genes that were differentially expressed in relapse and remission samples, we calculated average gene expression between the relapse and remission samples and subtracted gene expression values from relapse and remission samples for each gene. From this analysis, we chose the top 5 differentially expressed genes that were up regulated in the relapse and remission samples. We demonstrated differential expression of TNFAIP3 (Tumor Necrosis Factor Inducible Protein A20), which was higher in the remission sample as compared to relapse sample of a MOG-AAD patient (FIG. 3B). The other top genes identified to be up regulated during remission were HILPDA (Hypoxia Inducible Lipid Droplet-Associated), MXI-1 (MAX Interactor 1, Dimerization Protein), BNIP3 (BCL2 Interacting Protein 3), FAM162A (Family With Sequence Similarity 162 Member A); while genes up regulated during relapse included SNRPG (Small Nuclear Ribonucleoprotein Polypeptide G), EWSR1 (Ewing Sarcoma RNA Binding Protein 1), AL137058.2 (Clone-based Ensemble gene), HMGN1 (High Mobility Group Nucleosome Binding Domain 1), LRRC75A-AS1/SNHG29 (Small Nucleolar RNA Host Gene 29).

In order to validate our single cell RNA sequencing findings, we sought to evaluate the same MOG-AAD patient #1 with 3 longitudinal samples during remission (MOG-AAD #1.2), pre-relapse (MOG-AAD #1.3) and at a relapse (MOG-AAD #1.4) using DGE sequencing. DGE sequencing is a cost-effective method that offers low background noise, increased sensitivity and reproducibility. For this, PBMCs were stimulated with individual MOG peptides, p35-55, p119-130 and p181-195. Total RNA from CMC T cells was isolated and sequenced.

Broad Genomics Platform sequenced the libraries. Consistent with the single cell RNA sequencing findings, we found that gene expression of TNFAIP3 was decreased at both the pre-relapse and relapse sample compared to the remission sample. TNFAIP3 is known to be a regulator of NFκβ. We found that the gene expression of NFκβ1 was increased at relapse compared to remission time point, the inverse of TNFAIP3 expression (FIG. 3C). The other genes identified by single cell RNA sequencing, namely HILPDA, MXI-I, BNIP3 and FAM162A showed a similar trend by DGE sequencing. They were up regulated during remission as compared to pre-relapse and relapse time points. However, SNRPG, EWSR1, HMGN1 and LRRC75A-AS1, that were shown to be up regulated during pre-relapse time point by single cell RNA sequencing did not follow the same trend by DGE sequencing. Gene expression for AL137058.2 was below the detection limit by DGE sequencing.

To further confirm our RNA sequencing results, we decided to test another MOG-AAD patient #2 with 3 longitudinal samples, 1 at relapse (MOG-AAD #2.1) and 2 mycophenolate mofetil treated samples at remission (MOG-AAD #2.2 and MOG-AAD #2.3), using NanoString's differential gene expression platform. NanoString is a high throughput technique that allows simultaneous gene expression of more than 700 genes. We analyzed gene expression in CD4+ T cells, CD19+B cells and CD14+monocytes using the nCounter software. There was a distinct increase in the TNFAIP3 expression from CD4+ T cells in remission samples as compared to relapse sample thereby indicating that CD4+ T cells play an important role in TNFAIP3 regulation (FIG. 3D). In contrast, TNF-α expression from CD14+ monocytes, principal source of TNF-α in humans was increased in the relapse sample as compared to remission. CD4+ T cells followed a similar trend, where in TNF-α expression was higher at relapse as compared to remission samples.

To validate NanoString gene expression assay results, we isolated CD4+ T cells from 7 additional MOG-AAD patients with longitudinal samples (longitudinal samples n=5/7, relapse n=7 and remission n=8, total n=15). It also included MOG-AAD patient #2 with 3 longitudinal samples as previously used for NanoString gene expression assay. Quantitative real-time polymerase chain reaction (qPCR) was performed using FAM-labeled primer for TNFAIP3. GAPDH gene was used as an endogenous control to normalize for differences in the amount of total RNA in each sample. All values are shown as relative expression. The qPCR data replicated the results from NanoString gene expression assay. There was an increase in the relative expression of TNFAIP3 at remission time points and a decrease at relapse in the CD4+ T cells of MOG-AAD patient #2 (FIG. 3E). We next conducted grouped analysis of CD4+ TNFAIP3 expression levels from patients in relapse or remission states on disease modifying therapies, and samples from patients receiving high dose of corticosteroids, which are known to induce TNFAIP3 through binding of the glucocorticoid receptor. Grouped analysis comparing relapse samples (n=5), remission samples (n=5) and samples from patients treated with high dose of corticosteroids (n=4) showed a significant difference between the three groups (FIG. 3F) (Ordinary 1-way ANOVA; P=0.013).

### Example 3: Protein Expression Analysis Demonstrated Decreased TNFAIP3 Expression in MOG-AAD Patient at Relapse Timepoint

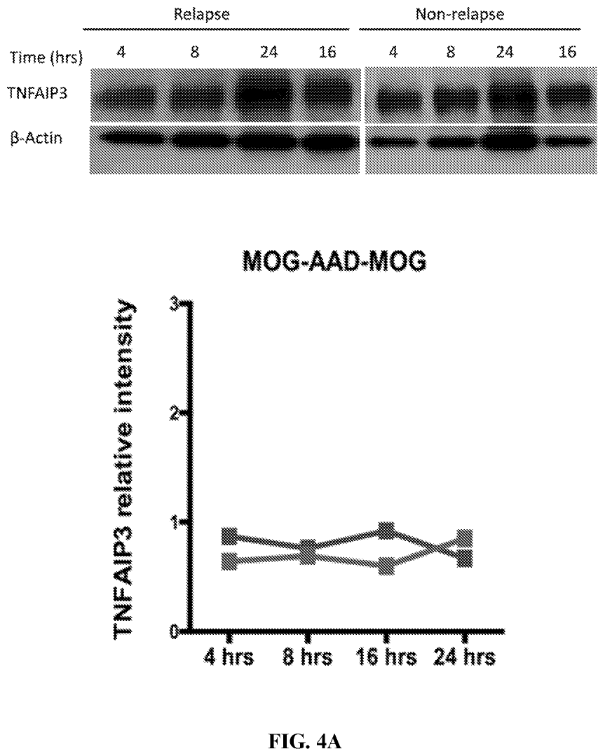

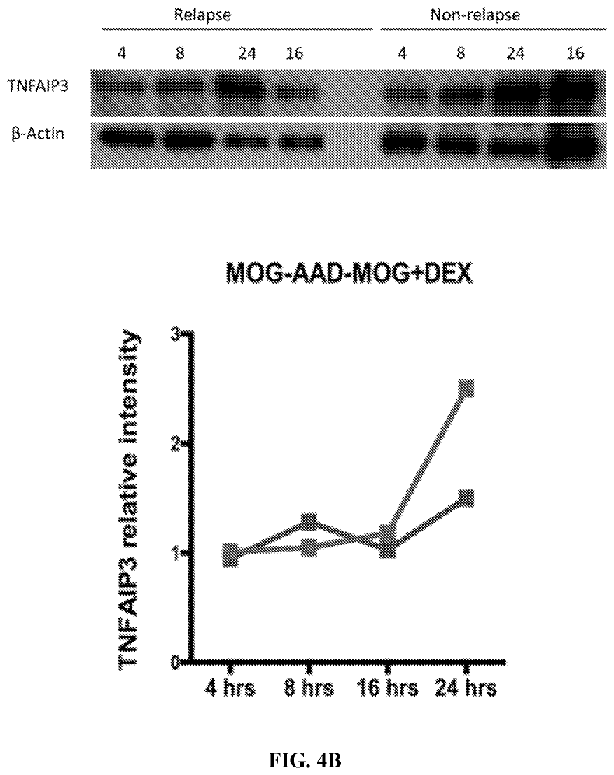

As the sequencing data on three independent platforms consistently suggested the differential expression of TNFAIP3 in MOG-AAD patients, protein expression of TNFAIP3 in whole PBMCs from an untreated MOG-AAD patient #3 (relapse MOG-AAD #3.1 and non-relapse MOG-AAD #3.2) was evaluated. Ligation of the TCR has been shown to induce TNFAIP3 expression and corticosteroids are commonly used as treatment for MOG-AAD which can aid in the resolution of relapses and potentially prevent new relapses. Hence, PBMCs were cultured with 2 conditions: MOG antigen stimulation (1 μg/ml) and MOG+dexamethasone stimulation (1 μg+100 nm) at 4 timepoints: 4, 8, 16 and 24 h. Addition of MOG peptide did not increase TNFAIP3 expression in relapse or non-relapse samples (FIG. 4A) but in the presence of both, MOG antigen and dexamethasone, which is a synthetic glucocorticoid, TNFAIP3 expression was comparable at 4, 8 and 16 h, however expression increased after 24 h of stimulation in the relapse sample as compared to non-relapse sample thereby partially rescuing the expression of TNFAIP3 in the relapse sample (FIG. 4B).

A dose response at lower and higher concentration of MOG antigen and dexamethasone stimulations in a MOG-AAD patient #2 (untreated, relapse MOG-AAD #2.1 and treated with mycophenolate mofetil, non-relapse MOG-AAD #2.3) were also studied. Here, PBMCs were cultured with 5 conditions: exvivo (unstimulated), MOG antigen stimulation (lower dose at 1 μg/ml and higher dose at 10 μg/ml) and MOG+dexamethasone stimulation (lower dose at 1 μg+100 nm and higher dose at 10 μg+1000 nm) at 3 timepoints: 4, 16 and 24 h. TNFAIP3 expression increased in presence of dexamethasone as compared to MOG antigen alone stimulation, more so in the non-relapse MOG-AAD sample as compared to relapse and with higher dose of MOG+dexamethasone. TNFAIP3 expression was also increased in the non-relapse MOG-AAD sample as compared to relapse MOG-AAD sample in the exvivo (unstimulated) condition.

Since TNFAIP3 is a well-known regulator of NFκβ, its correlation with NFκβ subunits p50 and p65 was studied in the same MOG-AAD patient #2 at a lower dose of MOG antigen stimulation (1 μg/ml) at 4 timepoints: 4, 8, 16 and 24 h. There was a negative correlation of TNFAIP3 expression with NFκβ subunits p50 and p65.

### Example 4: Serum Analysis Showed Decreased Levels of TNFAIP3 at Relapse and Increased Levels at Remission in MOG-AAD Patients and in Healthy Controls

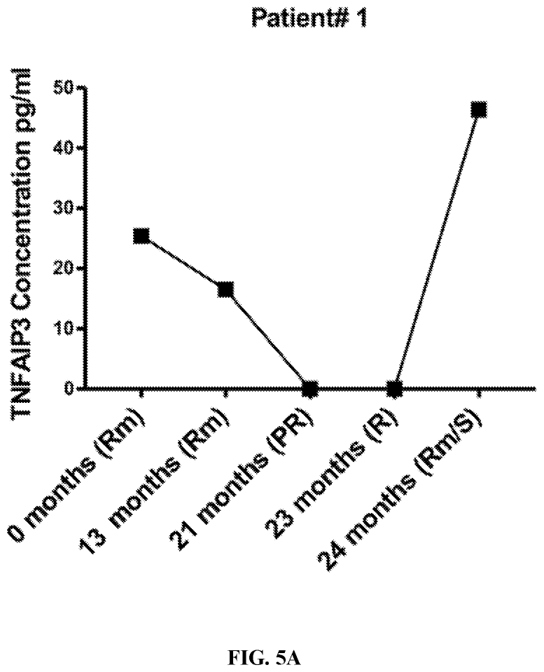

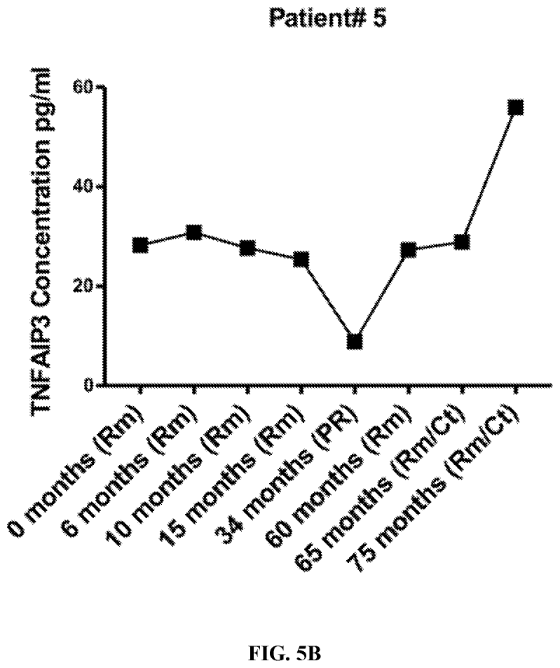

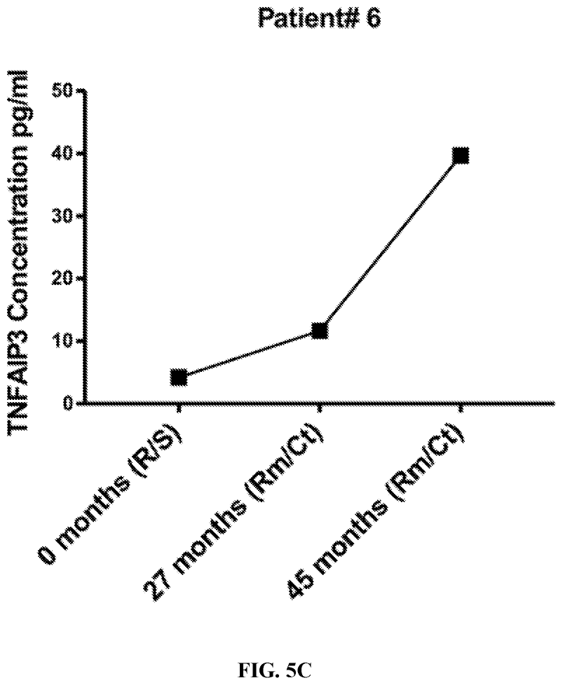

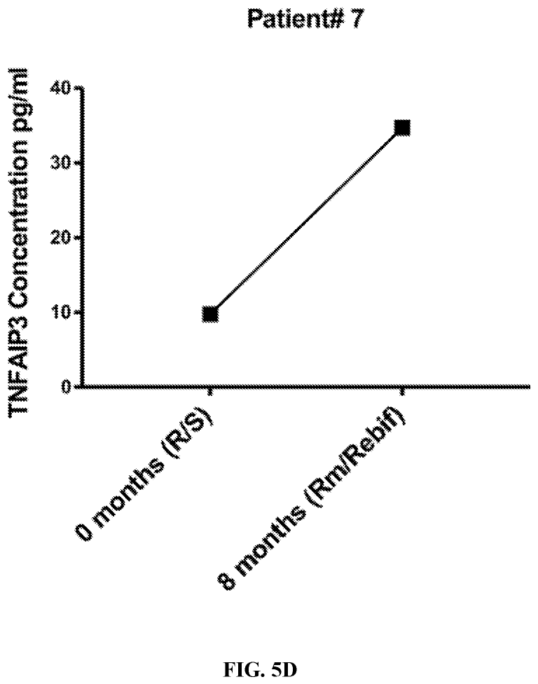

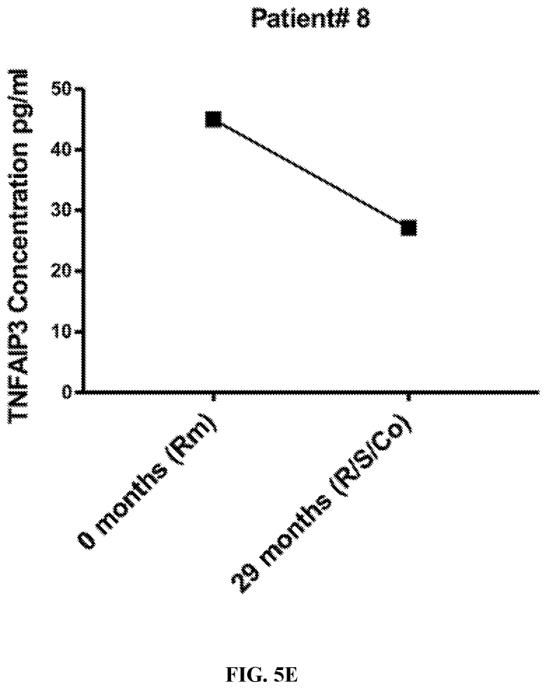

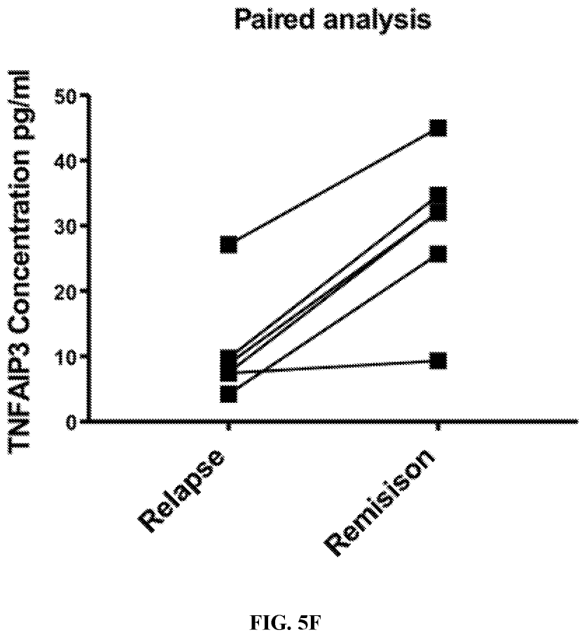

To confirm that alterations in TNFAIP3 transcription corresponded with translation into the TNFAIP3 protein, serum levels of TNFAIP3 in MOG-AAD patients were tested. A strong correlation of TNFAIP3 serum level decrement with the onset of relapse and increase following intravenous steroids in MOGAAD patient #1.1-1.5 was observed (FIG. 5A). Serum from other MOG-AAD patients, #5-8, with longitudinal samples showed similar trends (FIGS. 5B-5E). Mixed model paired analysis between relapse (n=8) and remission (n=22) samples from MOG-AAD patients (6 pairs) showed a significant reduction in TNFAIP3 levels in paired relapse samples as compared to remission samples (P=0.006) (FIG. 5F).

Further, using Nonlinear Mixed-Effects Models library in R (nlme), all available relapse n=10 and remission samples n=40 from MOG-AAD patients were evaluated and compared them with PHC n=44 samples. A significant reduction in TNFAIP3 serum levels in relapse samples as compared to remission samples (P=0.04) was found. There was also significant reduction in TNFAIP3 serum levels in relapse samples as compared to PHC (P=0.0001). This indicated that low levels of TNFAIP3 are associated with the onset and subsequent relapses in MOG-AAD patients whereas patients at remission and healthy controls show high levels of TNFAIP3.

In order to assess if there were other proteins associated with relapse in MOG-AAD, serum samples of 49 MOG-AAD patients (relapse samples n=10 and remission samples n=38) were evaluated using a multiplex inflammatory panel made up of 92 proteins. Data was presented as normalized protein expression (NPX) values. After adjusting for age, sex and treatments, 7 biomarkers were identified: FLT3 (Fms Related Tyrosine Kinase 3 Ligand, P=0.0061), CDCP1 (CUB Domain Containing Protein 1, P=0.008), IL12B (Interleukin 12B, P=0.0284), NTF3 (Neurotrophin 3, P=0.036), EIF4EBP1 (Eukaryotic Translation Initiation Factor 4E Binding Protein 1, P=0.04), KITLG (KIT Ligand, P=0.042), CCL2 (C-C Motif Chemokine Ligand 2, P=0.046) and CCL25 (C-C Motif Chemokine Ligand 25, P=0.05) that were significantly down regulated in relapse samples. However, these were no longer significant when corrected for multiple comparisons.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for analyzing a sample, the method comprising:
providing a sample from a subject, and
detecting tumor necrosis factor alpha-induced protein 3 (TNFAIP3) in the sample.

2. The method of claim 1, wherein detecting TNFAIP3 in the sample comprises detecting a level of TNFAIP3 protein in the sample.

3. The method of claim 2, wherein the level of TNFAIP3 protein is detected by an immunohistochemical assay, an immunoblotting assay, or a flow cytometry assay.

4. The method of claim 1, wherein detecting TNFAIP3 comprises detecting a level of TNFAIP3 nucleic acid.

5. The method of claim 4, wherein the level of TNFAIP3 nucleic acid is detected by a real-time reverse transcriptase PCR (RT-PCR) assay or a nucleic acid microarray assay.

6. The method of claim 1, wherein the sample is a blood sample or a serum sample.

7. The method of claim 1, wherein the sample is obtained from a subject having or suspected of having myelin oligodendrocyte glycoprotein antibody associated diseases (MOG-AAD).

8. The method of claim 7, wherein the MOG-AAD is selected from the group consisting of acute disseminated encephalomyelitis (ADEM), clinically isolated syndrome (CIS), optic neuritis (ON), recurrent forms of ADEM and ON, transverse myelitis (TM), and neuromyelitis spectrum disorder (NMO-SD).

9. The method of claim 1, further comprising administering the subject a treatment for MOG-AAD.

10. The method of claim 1, wherein the subject is a human patient or a non-human animal.

11. A method of diagnosing a subject as having myelin oligodendrocyte glycoprotein antibody associated diseases (MOG-AAD) or a relapse thereof, the method comprising:
providing a sample from the subject,
detecting a level of tumor necrosis factor alpha-induced protein 3 (TNFAIP3) in the sample;
comparing the level of TNFAIP3 in the sample to a reference level, and
diagnosing a subject as having MOG-AAD or a relapse thereof when the level of TNFAIP3 in the sample is lower than the reference level.

12. The method of claim 11, wherein detecting TNFAIP3 in the sample comprises detecting a level of TNFAIP3 protein in the sample.

13. The method of claim 12, wherein the level of TNFAIP3 protein is detected by an immunohistochemical assay, an immunoblotting assay, or a flow cytometry assay.

14. The method of claim 11, wherein detecting TNFAIP3 comprises detecting a level of TNFAIP3 nucleic acid.

15. The method of claim 14, wherein the level of TNFAIP3 nucleic acid is detected by a real-time reverse transcriptase PCR (RT-PCR) assay or a nucleic acid microarray assay.

16. The method of claim 11, wherein the sample is a blood sample or a serum sample.

17. The method of claim 11, wherein the sample is obtained from a subject having or suspected of having MOG-AAD or a relapse thereof.

18. The method of claim 17, wherein the MOG-AAD is selected from the group consisting of acute disseminated encephalomyelitis (ADEM), clinically isolated syndrome (CIS), optic neuritis (ON), recurrent forms of ADEM and ON, transverse myelitis (TM), and neuromyelitis spectrum disorder (NMO-SD).

19. The method of claim 11, further comprising administering the subject a treatment for MOG-AAD.

20. The method of claim 11, wherein the subject is a human patient or a non-human animal.


SEQUENCE LISTING (1 sequence)

SEQ ID NO: 1 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG